(19) United States
(12) Patent Application Publication
(10) Pub. No.: US 2003/0225425 A1
(43) Pub. Date: Dec. 4, 2003

(54) Devices and methods for interconnecting vessels

(76) Inventors: David Kupiecki, San Francisco, CA (US); Candice Danielle Pinson, Mountain View, CA (US); Stanley R. Conston, San Carlos, CA (US)

Correspondence Address: VASCONNECT, INC., 1460 MARIA LANE, SUITE 310, WALNUT CREEK, CA, 94596, US

(21) Appl. No.: 10/162,122

(22) Filed: Jun. 3, 2002

(51) Int. Cl. A61B 17/08

(52) U.S. Cl. 606/153

(57) ABSTRACT

Devices and associated methods for implanting or delivering devices within vessels, lumens, ducts or other tubular organs rapidly, safely and in a minimally invasive manner. The subject devices include a dilator/sheath assembly configured for operatively holding an anastomotic connector for subsequent delivery into target and graft vessels, etc. The subject methods involve the delivery of an anastomotic connector using the subject devices.

### TECHNICAL FIELD

The present invention is generally related to interconnecting body conduits. More particularly, the present invention is related to devices and methods for delivering and implanting devices for interconnecting body conduits such as blood vessels.

### BACKGROUND OF THE INVENTION

The human body has numerous vessels carrying fluid to essential tissues and areas for circulation or excretion. When vessels become damaged, severed or wholly occluded due to physiological problems or diseases, certain sections must be bypassed to allow for the free and continuous flow of fluids. Anastomosis is a procedure performed for the purpose of connecting different conduits together to optimize or redirect flow around a damaged or occluded portion of a vessel.

In the context of the peripheral vascular and/or the cardiovascular system, atherosclerosis, a common vascular disease, can cause partial blockage or complete occlusion of an arterial vessel, resulting in restricted blood flow and therefore compromised perfusion to the tissue served by the blood flow. In the case of an occluded or partially occluded coronary vessel, for example, an area of the heart's myocardium would be compromised, which can lead to a myocardial infarction, or other ischemic heart syndrome such as congestive heart failure. In the case of peripheral vascular atherosclerotic disease, occluded vessels lead to ischemic syndromes such as threatened limbs, stroke and other morbidities. In many cases, such a blockage or restriction in the blood flow leading to the heart or peripheral vessels can be treated by a surgical procedure known as an artery bypass graft procedure.

A bypass procedure involves the establishment of an alternate blood supply path to bypass a diseased section of a diseased or compromised artery. In the bypass procedure, the surgeon typically dissects one end of a source or “pedicled” artery (such as the internal mammary artery in the case of coronary artery bypass), or a free vessel member (typically the saphenous vein in the leg), to use as a graft conduit to bypass the obstruction in the affected artery to restore normal blood flow. The graft vessel is connected to the obstructed vessel by means of an anastomosis procedure wherein an opening in the graft vessel is sutured to the obstructed vessel at an arteriotomy site made within the obstructed vessel. A side-to-side anastomosis procedure involves the attachment of two vessels at incised locations (e.g., arteriotomies) within a side wall of each of the vessels. An end-to-side anastomosis procedure involves the attachment of two vessels at an incised location within a side wall of one of the vessels and at the transected end of the other vessel.

Other applications in which anastomosis is employed include the creation of an arterial to venous fistula for the purpose of either creating a dialysis access site, or, as an alternative means of creating arterial revascularization by “arterializing” a vein through creation of a conduit past the occlusive disease. The latter is often employed in treating peripheral vascular disease but is used in coronary applications as well.

The patency of the anastomosis is crucial to a successful bypass, both by acute and long-term evaluation. Patency may be compromised by technical, biomechanical or pathophysiological means. Among the technical and biomechanical causes for compromised patency (also termed restenosis) are poorly achieved anastomoses, whether induced by poor placement, trauma at the anastomosis site or biological responses to the anastomosis itself. Improperly anastomosed vessels may lead to leakage, create thrombus and/or lead to further stenosis at the communication site, possibly requiring re-operation and further increasing the risk of stroke. As such, forming the anastomosis is the most critical procedure in bypass surgery, requiring precision and accuracy on the part of the surgeon.

The current gold standard for forming the anastomosis is by means of suturing openings (natural or artificial) in the vessels together. Surgeons must delicately sew the vessels together being careful not to suture too tightly so as to tear the delicate tissue, thereby injuring the vessel which may then result in poor patency of the anastomosis. On the other hand, surgeons sometimes inadvertently suture too loosely or do not properly place the sutures so as to provide a continuous seal around the arteriotomy site, resulting in leakage of fluid from the anastomosis. In addition to creating a surgical field in which it is difficult to see, leakage of fluid from the anastomosis site can cause serious drops in blood pressure, acute or chronic. The loss of blood may cause other deleterious effects on the patient's hemodynamics that may even endanger the patient's life. In addition to the inherent inconsistencies in suture tightness, placement and stitch size and the lack of reproducibility, suturing an anastomosis can be very time consuming.

Advances in anastomotic instruments have been devised in the attempt to provide greater reproducibility of a precise anastomosis and to reduce the time that is required to complete an anastomosis and the necessary size of the surgical field. Many of these new instruments are stapling devices which deploy one or more staples at the anastomotic site in a single-motion action. While stapling techniques have been found to be successful in gastrointestinal procedures, due to the large size and durability of the vessels, it is less adequate for use in vascular anastomosis where the vessels are much smaller.

Moreover, the manufacturing of stapling instruments small enough to be useful for anastomosing smaller vessels, such as coronary arteries, is very difficult and expensive. As stapling instruments are typically made of at least some rigid and fixed components, a stapler of one size will not necessarily work with multiple sizes of vessels. This requires a surgeon to have on hand at least several stapling instruments of varying sizes. This may significantly raise the cost of the equipment and ultimately the cost of the procedure.

Stapling instruments and staples which are adapted to conform to the smaller sized vessels are difficult to maneuver and, thus, a great deal of time, precision, and fine movement is necessary to successfully approximate the vessel tissue. Often stapling or similar coupling devices require the eversion of the vessel walls to provide intima-to-intima contact between the anastomosed vessels. Everting may not always be practical especially for smaller arteries because of the likelihood of tearing when everted. Another factor which may lead to damage or laceration of the vessel and/or leakage at the anastomosis site is the variability of the force that a surgeon may use to fire a stapling instrument causing the possible over- or under-stapling of a vessel. Still other factors include the unintended inversion of the vessel edges and the spacing between staple points. Rectifying a poorly stapled anastomosis is itself a complicated, time-consuming process which can further damage a vessel.

The tension and/or compression forces exerted on the vessel walls as a result of suturing and stapling can result in damage to the vessel wall, even to the extent of causing tissue necrosis. Damage to the intima of a vessel is particularly problematic as it may inhibit the natural bonding process that occurs between the anastomosed vessels and which is necessary for sufficient patency. Furthermore, damaged vessel walls are likely to have protuberances that, when exposed to the blood stream, could obstruct blood flow or may produce turbulence which can lead to formation of thrombus, stenosis and possible occlusion of the artery.

As cardiac surgery is moving into less invasive procedures, surgical access is being reduced, forcing surgeons to work in constantly smaller surgical fields. These procedures are made more difficult due to the multiple characteristics that are unique to each anastomosis and to each patient. For example, the arteries' internal diameter dimensions are difficult to predict and the inside walls are often covered with deposits of stenotic plaque which creates the risk of dislodging plaque into the patient's blood stream during the anastomosis procedure. The resulting emboli in turn create a greater risk of stroke for the patient. The dislodgement of plaque is most likely to occur when the vessel wall undergoes trauma such as the puncturing, compression and tension exerted on the vessel by suturing and stapling. The vessel walls can also be friable and easy to tear, and are often covered with layers of fat and/or are deeply seated in the myocardium, adding to the difficulty of effectively and safely performing conventional anastomotic procedures.

Many of the drawbacks of the above mentioned anastomotic connectors and techniques have been obviated by recent technological advancements made by the assignee of the present invention. In particular, novel anastomotic connectors have been developed which avoid compression, tensioning and puncturing of the vessel tissue. Examples of such anastomotic connectors are disclosed in U.S. Pat. Nos. 6,165,185 and 6,251,116, and in U.S. patent application Publication No. US-2001-0044631-A1; all of which are herein incorporated by reference. These devices include at least one flexible member in the form of a sheet, membrane or flange which is adapted to conform to and seal with an inner surface or circumference of a vessel into which it is delivered. The flexible member is adapted to utilize only the internal vessel pressure, e.g., blood pressure, exerted thereon to form a substantially fluid-tight seal with the inner surface of the conduit whereby substances within the vessel are prevented from leaking from the artificial opening under normal physiological conditions. As such, these devices obviate the need to compress, puncture or place tension on the vessel tissue and reduce many of the risks associated with prior anastomotic and closure devices. Another advantage of these flexible devices is that they can be made from materials which are biodegradable or bioresorbable, such as degradable hydrogels, polymers, protein cell matrices, plant or carbohydrate derivatives (sugars), and the like.

Unlike staples, clips, sutures and the like, which often require the surgeon to employ many components for their delivery and implantation in the body, the flexible flanges or membranes may be implanted manually by a surgeon. As such, the use of cumbersome and complicated instrumentation necessary for implanting the devices may be avoided; however, the time and skill required for manual implantation may present difficulties to the surgeon. It is further desirable to minimize tissue trauma and reduce the size of the surgical opening, which necessitates the use of minimally invasive delivery instrumentation and techniques. Furthermore, it would be additionally beneficial and desirable if such instrumentation was easier to use, required fewer components, reduced the procedure time, reduced the risk of improper alignment between the conduits, and minimized the risk of leakage, tearing and damage at the anastomosis site. It is additionally desirable to provide such delivery devices in which a single configuration may be employed with a variety of configurations of anastomotic connectors, including both side-to-side and end-to-end devices. Further, it would be highly advantageous if such delivery devices were usable for both proximal anastomosis applications, e.g., a graft vessel to the aorta, and distal anastomosis applications, e.g., a graft vessel to a native vessel at a locatin downstream of the stenotic lesion within the native vessel.

These and other objects, advantages, and features of the invention will become apparent to those persons skilled in the art upon reading the details of the methods and systems of the present invention which are more fully described below.

Relevant Literature

U.S. Patents of interest include: U.S. Pat. Nos. 6,113,612; 6,113,611; 6,090,136; 6,068,656; 6,068,637; 6,063,114; 6,056,762; 6,036,705; 6,036,704; 6,036,703; 6,036,702; 6,030,392; 6,026,814; 6,007,576; 6,007,544; 6,001,123; 5,961,545; 5,948,018; 5,921,995; 5,916,226; 5,904,697; and 4,214,586. Also of interest are the following PCT publications: WO 00/24339; WO 99/65409; WO 99/48427; WO 99/45852; WO 99/08603; WO 98/52474; WO 98/40036; WO 97/31591 and WO 97/31590.

### SUMMARY OF THE INVENTION

The present invention provides devices and associated methods for implanting or delivering devices within vessels, lumens, ducts or other tubular organs rapidly, safely and in a minimally invasive manner. These devices and methods are particularly helpful in surgical procedures involving the anastomosis of small vessels or the like within a limited surgical access field. A single configuration of the delivery or implantation device of present invention may be employed with a variety of embodiments of anastomotic connectors.

The present invention is useful for delivering side-to-side and end-to-side anastomotic connectors to join any two (or more) vessels together such that fluid communication is established between the lumens of the two or more joined vessels, where representative types of vessels include, but are not limited to, vascular vessels and other vessels of the body, where one of the vessels may be a synthetic vessel or graft vessel from a donor, e.g., autograft or allograft. The present invention is particularly useful for joining vessels in coronary artery bypass graft procedures (CABG), in peripheral vascular bypass graft procedures, such as femoropopiteal (Fem-Pop) bypasses, and to form arterial-venous fistulas.

These and other objects, aspects, advantages and features of the invention will become apparent to those skilled in the art upon reading this disclosure in combination with the accompanying figures.

### BRIEF DESCRIPTION OF THE FIGURES

To facilitate understanding of this disclosure, the same reference numerals have been used (where practical) to designate similar elements that are common to the Figures. Some such numbering has, however, been omitted for the sake of clarity.

FIG. 1 is a perspective view of a side-to-side anastomotic connector which is implantable by means of the delivery devices of the present invention.

FIG. 2 is a top planar view of another side-to-side anastomotic connector which is implantable by means of the delivery devices of the present invention.

FIG. 3 is a perspective view of an end-to-side anastomotic connector which is implantable by means of the delivery devices of the present invention.

FIGS. 4A and 4B illustrate a delivery device of the present invention.

FIG. 5A illustrates an enlarged view of the distal end of the delivery device of FIGS. 4A and 4B having the side-to-side anastomotic connector of FIG. 2 operatively loaded thereon.

FIG. 5B illustrates an enlarged view of the distal end of the delivery device of FIGS. 4A and 4B having the end-to-side anastomotic connector of FIG. 3 operatively loaded thereon.

FIGS. 6A-6D illustrate the steps of using the delivery device of FIGS. 4A and 4B to deliver the side-to-side anastomotic connector of FIG. 5A according to a method of the present invention.

FIGS. 7A-7C illustrate the steps of using the delivery device of FIGS. 4A and 4B to deliver the end-to-side anastomotic connector of FIG. 5B according to a method of the present invention.

### DETAILED DESCRIPTION OF THE PREFERRED EMBODIMENTS

Before the present invention is described in such detail, it is to be understood that this invention is not limited to particular variations set forth herein as various changes or modifications may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events or steps which is logically possible. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

In further describing the subject invention, the anastomotic devices which may be used with the present invention are described first. Next, a description of the subject delivery devices and systems is provided followed by a description of the methods of using them. Finally, a review of the kits of the present invention which include the subject delivery systems and devices for performing the subject methods is provided.

In the following description, the present invention as used in anastomotic applications will be described in the context of joining two vessels wherein at least one of the vessels is the target vessel to be bypassed such as a coronary or peripheral vessel. The other vessel is a graft vessel which may be pedicled or segmented from its native location. However, such exemplary application is not intended to be limiting and those skilled in the art will appreciate that the subject devices, systems and methods are useful for the joining of such vessels in alternate configurations as well as the joining of other types of conduits and structures.

Anastomotic Connectors

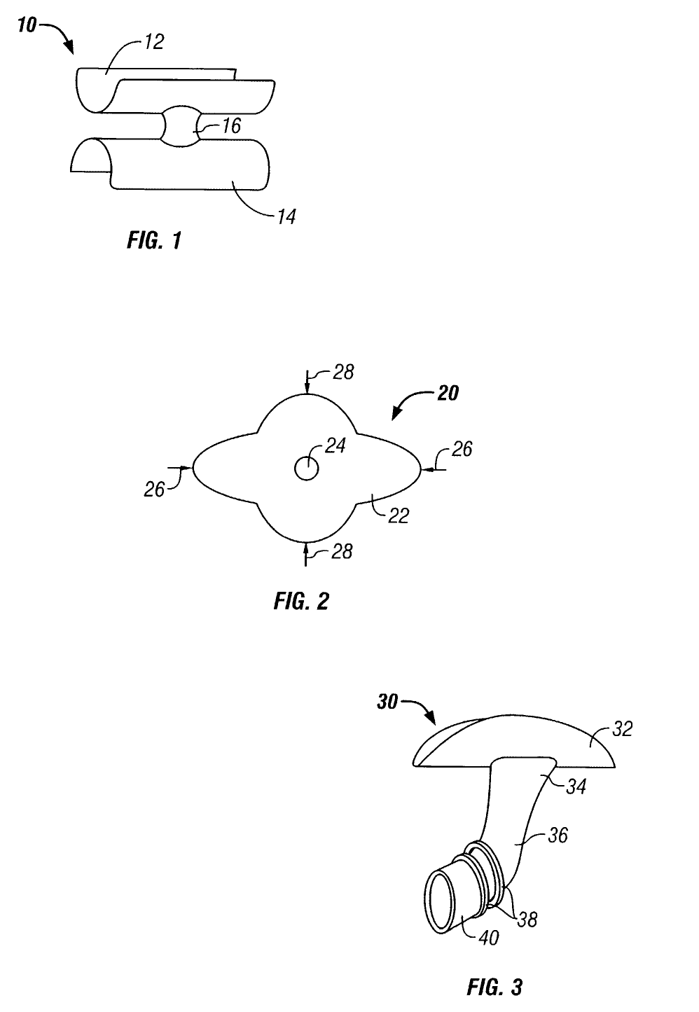

FIGS. 1-3 illustrate various embodiments of the anastomotic connectors generally described above which are suitable for use with the present invention. Such devices are described in detail in U.S. Pat. Nos. 6,165,185 and 6,251,116, and in U.S. patent application Publication No. US-2001-0044631-A1. While the subject invention is especially useful for delivering the anastomotic connectors disclosed in these patents, it will be obvious to those skilled in the art that the subject devices and methods herein described may be employed with variations of these anastomotic connectors. As such, reference to specific embodiments of anastomotic connectors is solely for purposes of describing the subject invention and is not in any way intended to limit the scope or the function of the subject invention.

While reference can be made to the above-referenced patents for a detailed description of anastomotic connectors usable with the present invention, a brief description is herein provided for purposes of convenience. The delivery devices of the present invention may be used with both side-to-side and end-to-side anastomotic connectors and procedures. A side-to-side anastomosis procedure involves the attachment of two vessels at incised locations (e.g., arteriotomies) within a side wall of each of the vessels. An end-to-side anastomosis procedure involves the attachment of two vessels at an incised location within a side wall of one of the vessels and at the transected end of the other vessel.

Common to the anastomotic connectors of the present invention are two members which are in fluid communication with each other. Each device comprises at least one flexible member in the form of a sheet, membrane or flange. The devices configured for forming a side-to-side anastomotic connection include a second flexible membrane wherein a flow opening or channel resides between the members such that they are in fluid communication. Those connectors configured for forming an end-to-end anastomotic connection have a second member having a tubular configuration wherein the lumen of the tubular member extends from a flow opening in the flexible member.

In either configuration, the flexible members are adapted to conform to and seal with an inner surface or circumference of a vessel into which it is delivered. Furthermore, the flexible member is adapted to utilize the internal vessel pressure exerted thereon to form a substantially fluid-tight seal with the inner surface of the conduit whereby substances within the vessel are prevented from leaking from the artificial opening under normal physiological conditions. More particularly, the flexible member has first and second surfaces. The first or lumen-facing surface is adapted to utilize the internal conduit pressure exerted thereon to form a substantially fluid-tight seal between the second or vessel-contacting surface and an inner wall or circumference of the vessel. Thus, upon deployment of the flexible member into a vessel, the member conforms to the interior walls of the vessel to provide a sealing contact and sufficient physical stability to the device to prevent displacement from the vessel. Moreover, the substantially fluid-tight seal is formed without compressing, tensioning or puncturing the vessel wall.

The flexible members are constrictable (such as by bending or folding) to a size sufficient to fit through the artificial opening and are expandable to be securely and permanently self-retained within the vessel upon implantation. The flexible members comprise relatively thin walls, thus minimally interfering with fluid flow within the interconnected vessels. The intravascular pressure against the underside of the flexible member secures the member against the inside vessel wall thereby preventing leakage from the anastomosis site. Additionally, the configuration of the flexible members is such that it provides an element of passive force when deployed within the vessel so as to pull the two vessels together for better sealing and healing of the vessel walls.

In certain embodiments the flanges have constant diameters about their circumference (e.g., circular) or the same length and width dimensions (e.g., square). In other embodiments, the flanges have varying diameters (e.g., elliptical, oval) or lengths and widths (e.g., rectangular), wherein the flanges have a major axis, i.e., a longer axis, and a minor axis, i.e., a shorter axis. In any embodiment, the flexible membranes are sufficiently flexible and compliant to be folded about any axis defined by the membranes, as well as to be folded about an axis which is defined by the flow opening or channel, which may be substantially perpendicular to the surface of the flange or at angle to the flange. Such flexibility facilitates implantation of the anastomotic connectors with the devices of the present invention.

Upon release of the membranes from a constricted or folded condition, each membrane subsequently conforms to the interior walls of a conduit to provide a sealing contact along the contact surface of the membrane. Once deployed within the conduits, the sealing contact and stiffness properties of the flanges provide sufficient physical stability to the device to prevent displacement from the respective vessels.

The flexible flanges may have a variety of different configurations, shapes, thickness(es), surface areas, lengths and widths (or diameters). Useful configurations include, but are not limited to, partial cylinders or generally planar configurations having circular elliptical, stared, petaled or rectangular shapes, or combinations of these configurations.

Each flange or membrane includes an opening through its thickeness which provides a pathway through which fluid can be transported between anastomosed conduits. More specifically, the flow opening provides a location of permanent connection between the two members of the anastomosis device, whether a side-to-side or an end-to-side device, and thus, establishes fluid communication between the vessels connected by the implanted device.

Generally, the size and shape of the flexible members are dependent on the size (i.e., the circumference or diameter) and shape of the bodily lumen into which it is to be used. For example, larger segments may be preferable when performing a proximal anastomosis to an aorta, or when anastomosing peripheral (e.g., in the leg) or abdominal vessels while smaller segments are more appropriate for interconnecting coronary arteries and veins. Also, the length or width (or diameter) dimensions or both, may be dictated by the length of the incision or arteriotomy within the lumen or vessel into which the segment is to be placed.

The anastomotic connectors may be made of biodegradable or bioresorbable materials or non-resorbable materials. Suitable bioresorabable materials include but are not limited to degradable hydrogels, polymers such as lactides/glycolides or PHAs; protein cell matrices, plant, carbohydrate derivatives (sugars), and the like. Suitable non-resorbable materials include but are not limited to polymers and elastomers such as silicones, fluoropolymers, polyolephins or polyurethanes might also be used. In addition, the anastomotic connectors may be fabricated from composites of two or more different types of materials, etc, e.g., the device may be fabricated from a blood impermeable membrane attached to a structural article or scaffold. In addition to being adequately biocompatible, the material(s) have appropriate mechanical properties for facilitating insertion, retention and sealing of the members within the vessels. Additionally, the anastomotic connectors may be made of any suitable autologous, allo- and xeno-graft biomaterials.

Referring now to the Figures, specific embodiments of anastomotic connectors are illustrated which are usable with the present invention. Side-to-side anastomotic connector 10 of FIG. 1 includes both a first portion or flexible member, membrane or flange 12 and a second portion or flexible member, membrane or flange 14 connected by a flow channel 16 which extends between the two flanges to provide fluid communication between the vessels into which flanges 12 and 14 are inserted. In this embodiment, each flange has a rectangular contact surface which, when in a constricted condition along the longitudinal axis of the flange, has a semi-cylindrical configuration.

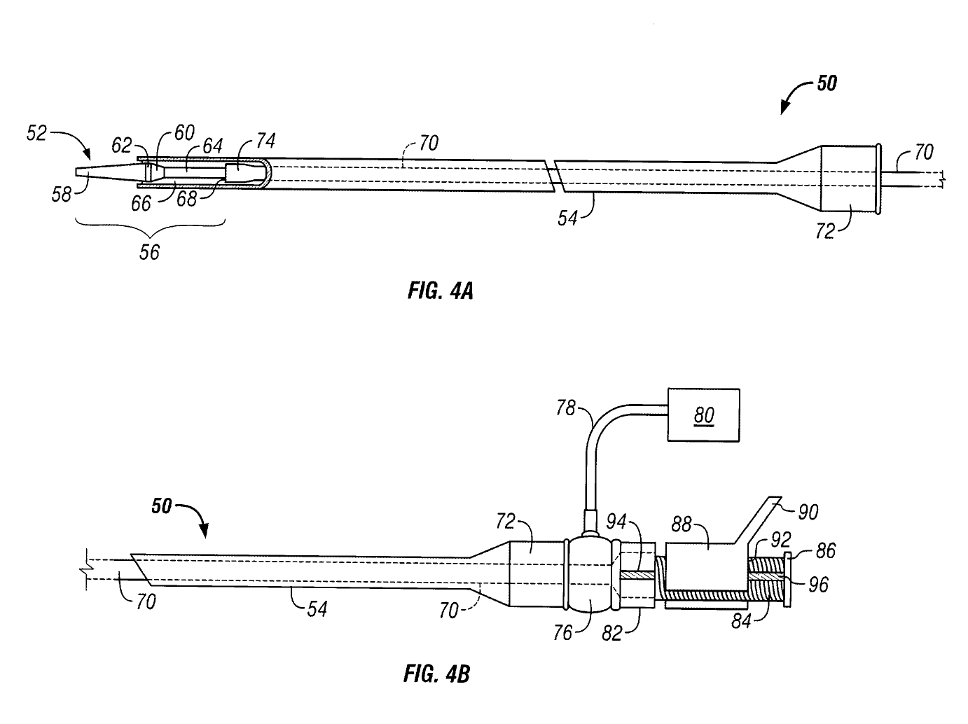

FIG. 2 illustrates a top planar view of another side-to-side anastomotic connector 20 having a first portion or flange 22 having a pedal configuration and a flow opening 24 and a second portion or flange and associated flow opening (neither of which are shown) which correspond in size and shape to flange 22 and opening 24, respectively. Between the flow openings extends a flow channel (not shown) similar to the flow channel of FIG. 1. As mentioned above, the side-to-side or end-to-end distances of flange 22, designated by arrows 26 and 28, may be the same or differ from each other. In certain embodiments, the flanges may have a major axis, such as defined by arrows 26, and a minor axis, such as defined by arrows 28. The flanges are bendable or foldable about either axis, and thus, device 20 may be implanted in either folded configuration as required by the surgical application.

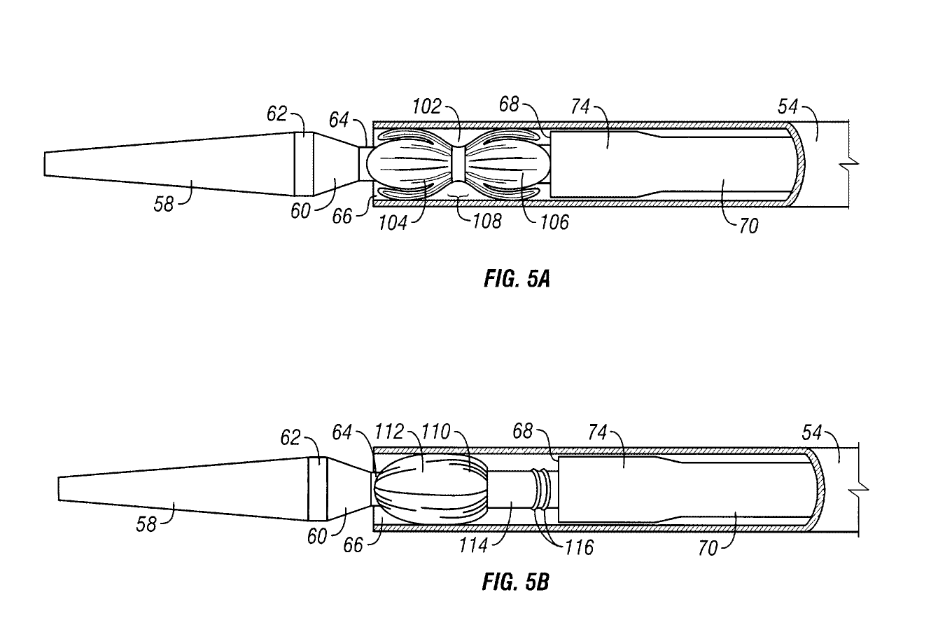

FIG. 3 illustrates an end-to-side anastomotic connector 30 having a first portion or membrane or flange member 32 having an oval shape and a second portion or tubular member 36 joined together at a flow opening, defined externally by juncture 34, analogous to that found in the side-to-side devices described above. The flange member 32 of the end-to-side device has the same or similar properties and advantages as described above with respect to the flange members of the side-to-side device. Flange 32 is shown as a partial cylinder having an elliptically shaped contact surface.

Tubular member 36 may be normal to, or positioned at an angle relative to, the surface of flange member 32. Tubular member 36 is designed to fit inside of the transected end of a graft vessel that is to be joined to the side wall of a host vessel. The length of tubular member 36 typically ranges from about 10 mm to about 20 mm. The outer diameter of tubular member 140 has a dimension that approximates the inner diameter of the graft vessel to be attached, and therefore is typically in the range from about 2 mm to about 6 mm, and more typically from about 3 mm to about 5 mm and may be cylindrical or conical in aspect. Optionally, tubular member 36 has a vessel securement means 38 for further securing tubular member 36 within a graft vessel. As shown here, vessel securement means 38 is in the form of two parallel rings surrounding the circumference of tubular member and appropriately positioned vis-à-vis the host vessel, another component of the securement means such as a cuff or ring (not shown) may be temporarily or permanently positioned about the graft vessel and within the spacing formed by the parallel rings.

Delivery Devices of the Present Invention

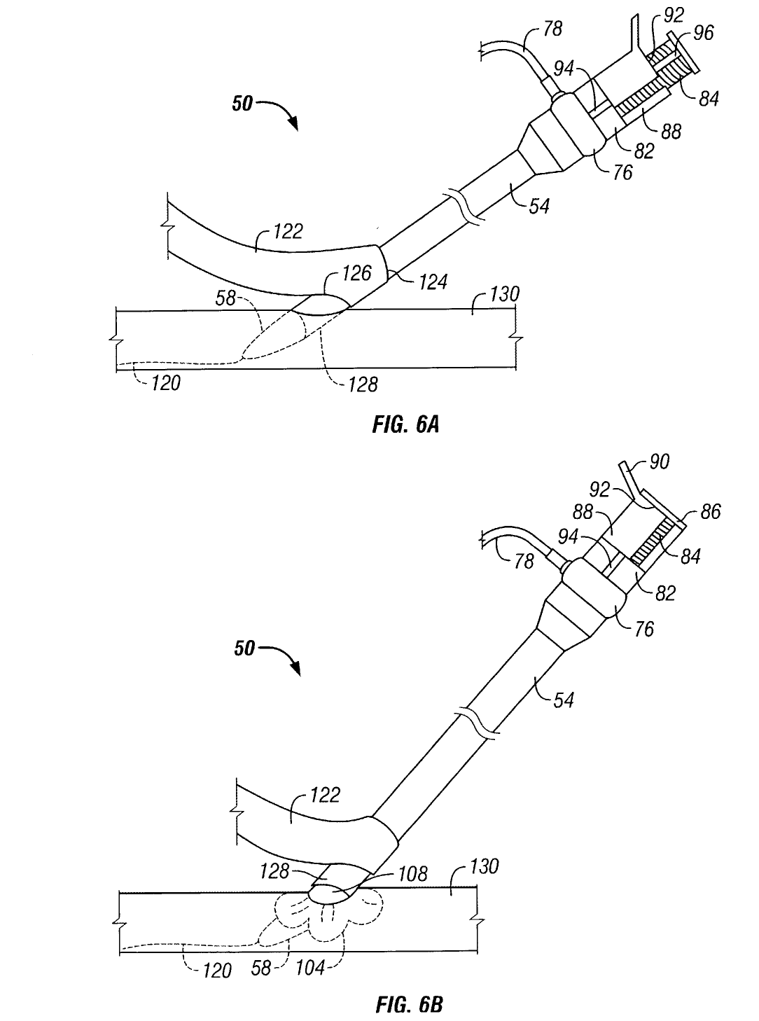

Referring now to FIGS. 4A and 4B, there is illustrated an exemplary embodiment of a delivery device or assembly 50 of the present invention. As shown in FIG. 4A, device or assembly 50 includes an introducer or dilator member 52 slideably engaged and translatable within the lumen of sheath 54. Dilator member 52 has small diameter guide wire lumen extending through its length for accommodating a guide wire (not shown). Dilator 52 has a primary shaft portion 70 which extends through the proximal end 72 of sheath 54 and a distal end portion 56. At least distal end portion 56 is relatively flexible so as to be manipulated to optimally deliver an anastomotic connector to within a vessel, but is sufficiently rigid to facilitate dilatation of the incision or arteriotomy site within which the anastomotic connector is to be delivered.

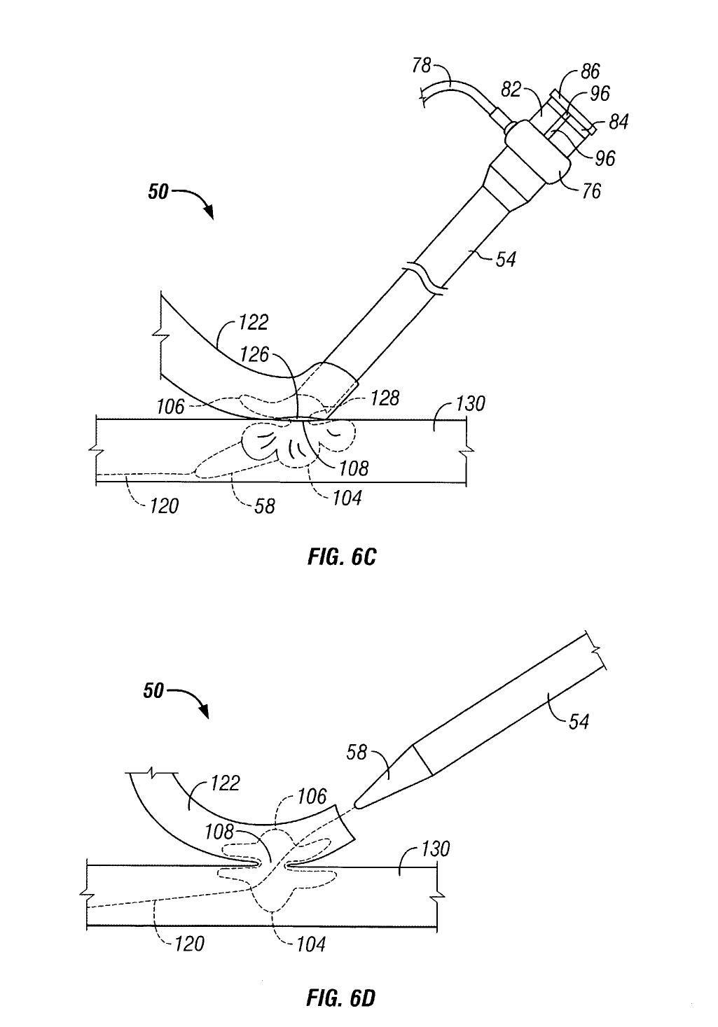

Distal end portion 56 includes a distally tapered tip or leading surface 58, a transition portion 62 and a proximally tapered shoulder 60. Proximal to tapered shoulder 60 is a necked down or recessed central shaft portion 64 which is defined at its proximal end by a ledge or shoulder 68 at the distal end 74 of primary shaft portion 70. Recessed shaft portion 64 thus defines an annular space or recess within the dilator shaft. When dilator member 52 is disposed within sheath 56, this annular space defines a cylindrical chamber or space 66 having length and diameter dimensions and defining a volume sufficient to operatively retain an anastomotic connector loaded thereon as illustrated in FIGS. 5A and 5B. In order to accommodate an anastomotic connector, recessed central shaft portion 64 has a length in the range from about 8 mm to about 15 mm, and more typically in the range from about 10 mm to about 12 mm; and an outer diameter in the range from about 1.3 mm to about 8 mm, and more typically in the range from about 2 mm to about 4 mm.

In FIG. 5A, a side-to-side anastomotic device 102 having a configuration similar to that of device 20 of FIG. 2, has been operatively loaded onto the dilator by inserting tapered tip 58 through the fluid channel 108 of device 102, and positioning connector 102 over central shaft portion 64. Flexible members 104 and 106 are then constrained compressed or folded against shaft portion 64, and dilator 52 is pulled within shaft 54 until at least the entirety of device 102 is covered and retained within chamber 66.

FIG. 5B illustrates an end-to-side anastomotic device 110 similar to that of device 30 of FIG. 3 operatively loaded within chamber 66. Tapered tip 58 is inserted into the end of tubular member 114 having securement rings 116, and through the fluid opening within flexible member 112, and device 110 is slid over the dilator until wholly positioned over necked down shaft portion 64. Flexible member 112 is then constrained compressed or folded against shaft portion 64, and dilator 52 is pulled within shaft 54 until at least the entirety of device 110 is covered and retained within chamber 66.

FIG. 4B illustrates the componentry of the dilator and sheath assembly 50 at its proximal end. At proximal end 72 of sheath 56 is a hub 76 from which extends an infusion line or tube 78 which is in fluid communication with a source of saline 80 or other fluid. Proximal to hub 76 is a sheath cap 82 having internal elastomer seal (not shown) and which is internally threaded to engage with a proximal end portion 84 of dilator shaft 70. Dilator proximal end portion 84 has an end cap 86 having a diameter greater than end portion 84. Also provided about end portion 84 is a dilator stop 88 having a stopping surface 92 and a split sleeve configuration so as to be easily removed from threaded end portion 84 when pulling on tab 90. Sheath cap 82 and dilator proximal end portion 84 each have an alignment strip 94 and 96, respectively, to assist the physician or user in properly aligning dilator 52 within shaft 54 in order to ensure proper positioning of the anastomotic connector upon deployment within a vessel.

Dilator 52 has an overall length which is greater than that of sheath 54 such that the distal end portion 56 of dilator 52 can be extended beyond the distal end of sheath 54. Their respective lengths and other dimensions will depend on the application at hand, i.e., whether the delivery procedure is performed through a conventional surgical incision or a small port, or performed percutaneously (a catheter-based approach). Generally, however, the length of dilator 52 ranges from about 5 cm to about 75 cm, and more typically from about 15 cm to about 35 cm. The length of sheath 54 ranges from about 5 cm to about 60 cm, and more typically from about 10 cm to about 30 cm. Primary shaft portion 70 and transition portion 62 have a diameter from about 1.5 mm to about 6 mm, and more typically in the range from about 2.3 mm to about 4 mm. Such diameter dimension is usually at least 1 mm larger than the diameter of recessed central shaft portion 64. Sheath 54 has an internal diameter which is generally slightly greater, e.g., from about 0.001 mm to about 0.01 mm, than the outer diameter of primary shaft portion 70, or is otherwise sufficiently greater to accommodate a graft vessel coaxially positioned about dilator 52 or a portion thereof when used, for example, for an end-to-side anastomosis application. The outer diameter of sheath 54 generally ranges from about 0.2 mm to 1 mm greater than the diameter of the primary shaft portion 70.

Dilator/sheath assembly 50 may further include viewing means (not shown), such as an endoscope, associated with it to facilitate visualization by the physician of the working space. Such is particularly helpful if performing the procedure through a thoracotomy, mini-thoracotomy, mini-sternotomy or through an access port formed in the patient's chest.

Methods of the Present Invention

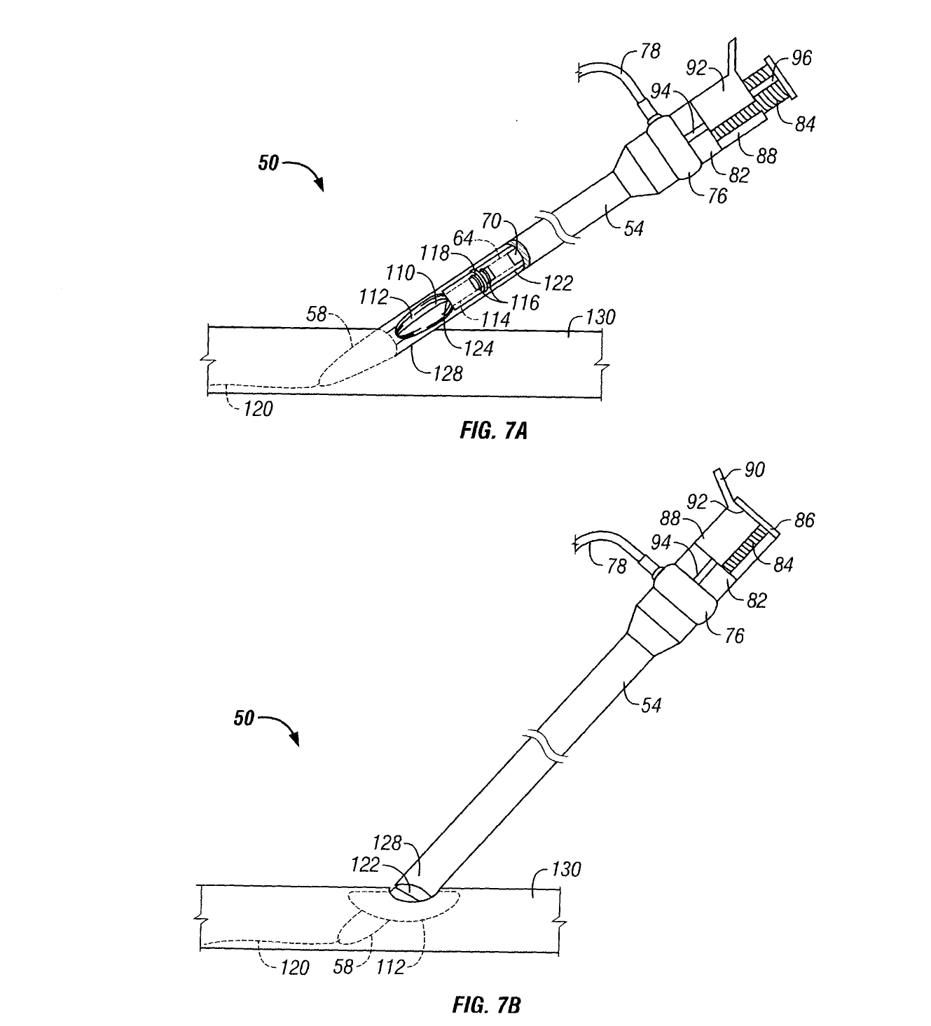

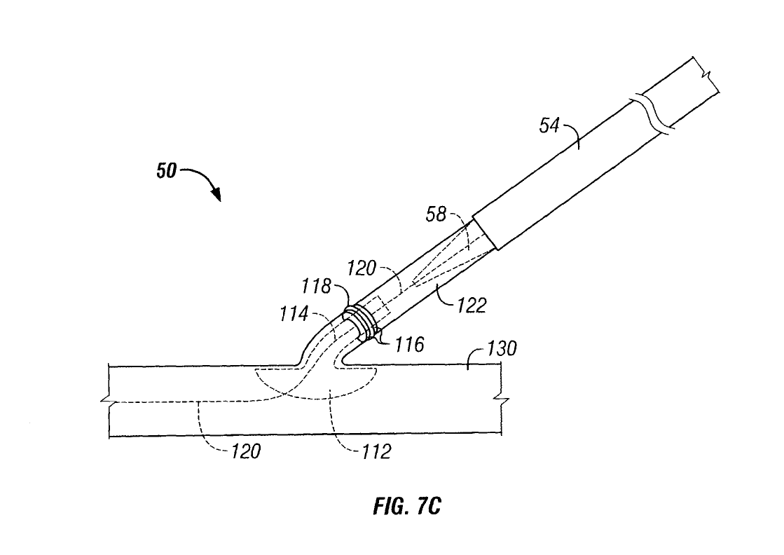

The subject methods are now described in detail with reference to FIGS. 6A-6D and 7A-7C. FIGS. 6A-6D illustrate the steps for delivering and implanting the side-to-side anastomotic connector of FIG. 5A to join a graft vessel with a target vessel. FIGS. 7A-7C illustrate the steps for delivering the end-to-side anastomotic connector of FIG. 5B to join a graft vessel with a target vessel.

Examples of suitable applications of the subject methods include but are not limited to coronary artery bypass grafting, peripheral artery bypass grafting, and the formation of arteriovenous fistulae.

The graft vessel may be a pedicled vessel requiring only distal attachment to the target vessel or may be a segmented vessel which requires both proximal and distal attachment. The subject devices and methods may be used to perform both proximal and distal anastomosis of the same graft vessel wherein the proximal procedure and the distal procedure may be performed in any order. For example, a segmented graft vessel may be anastomosed proximally to a blood supply vessel, such as the aorta, using either a side-to-side or an end-to-side device. The same vessel may then be anastomosed distally to the target vessel using either type of device.

The subject methods may be employed in an open surgical approach in which the physician directly visualizes the surgical field or in a less invasive approach wherein the physician must use an endoscope or the like to visualize the surgical field. Such less invasive methods may be performed through a small incision or port, or intravascularly wherein the subject delivery devices are configured as catheters.

The subject method begins by establishing access to the target vessel. Such may be accomplished by a small incision, i.e., an arteriotomy, made in the target vessel or by the Seldinger technique or a modification thereof. With the Seldinger technique, a small gauge needle is introduced through the wall of the target vessel, e.g., a coronary artery, and a guide wire is introduced through the needle and delivered to within the target vessel. After proper placement of the guide wire, the needle is withdrawn and the distal end of the guide wire is left in place within the target vessel. The remainder of the method steps is now described separately for side-to-side anastomosis and end-to-side anastomosis.

Side-to-Side Anastomosis

Referring specifically to FIGS. 6A-6D, a side-to-side anastomosis, either proximal or distal, is now described. Prior to introducing the subject delivery device 50 over the guide wire 120, a graft vessel 122 having a transected end 124 is provided. A small opening 126 is made within the side wall of graft vessel 122, either proximate to or at the end opposing transected end 124, with enough length there between such that the vessel can be tied off manually or with surgical clips.

As shown in FIG. 6A, with the anastomotic connector loaded on dilator 52 and fully contained within chamber 66, tapered distal end 58 of dilator 52 is inserted into graft vessel 122 through transected end 124 and back out of graft vessel 122 through side opening 126. Graft vessel 122 is then positioned over the distal end 128 of shaft 54. Once graft vessel 122 is operatively engaged with assembly 50, the proximal end of the guide wire 120 is inserted into the guide wire lumen of the dilator at the tapered distal end 58. Delivery device 50 is then delivered over the guide wire 120 to target vessel 130, at which point delivery device 50 is advanced a selected distance such that the tapered distal end 58 of dilator 52 is caused to penetrate through the wall of the target vessel 130. At this point, it should be noted that throughout the various steps of the subject methods, saline from source 80, when allowed to flow into hub 76 and then into sheath 54, may be used to create a positive pressure within sheath 74 thereby helping to maintain hemostasis of the target vessel, i.e., preventing the escape of blood from the opening formed therein. Additionally, the internal seal within sheath cap 82 prevents the fluid from escaping proximally from sheath 54, thereby further maintaining the positive pressure within sheath 54.

Upon distal end 58 being fully inserted within target vessel 130, dilator 52 is advanced distally through shaft 54 by pushing on threaded end portion 84 of dilator 52 a selected distance until end cap 86 is caused to abut stop surface 92 of dilator stop 88. During such advancement, proper alignment of dilator 52 is ensured by aligning alignment strip 96 with alignment strip 94 of sheath cap 82. Alternatively, sheath 54 may be retracted in a proximal direction a selected distance wherein the position of dilator 52 is static. The distance advanced by dilator 52 or retracted by sheath 54 causes a sufficient portion of chamber 66 to be exposed such that the distal flexible member 104 of the anastomotic connector loaded within chamber 66 is allowed to deploy within target vessel 130, as illustrated in FIG. 6B. By deployment of the flexible member 104, it is meant that the petals of flange member 104 expand from their constrained, compressed or folded condition to engage with the internal wall of target vessel 130.

At this point, graft vessel 122 is positioned such that the edge of its side wall opening 126 substantially engages with or appositions against the edge of the opening formed within target vessel 130. The position of graft vessel 122 may be adjusted manually, if possible, or by an elongated instrument. Next, dilator stop 88 is removed from the proximal end portion 84 of dilator 52 by pulling on tab 90. Dilator 52 is again advanced a selected distance in a distal direction and in proper alignment until its end cap 86 abuts against sheath cap 82. Alternatively, sheath 54 is again retracted a selected distance in a proximal direction while ensuring proper rotational alignment of dilator 52 within sheath 54. This secondary or additional advancement or retraction exposes the remainder of chamber 66 thereby allowing deployment of the second or proximal flange member 106, as illustrated in FIG. 6C. Such deployment of second flange member 106 is the same as that described with respect to the deployment of the first or distal flange member 104 except that the petals of flange member 106 expand against the internal wall of graft vessel 122. The respective openings of graft vessel 122 and target vessel 130 now encircle fluid channel 108 of the anastomotic connector and are caused to be pulled together to maintain contact between their respective edges. Preferably, the endothelial linings of the vessels are in intimal contact with each other so as to promote natural tissue bonding between them.

Finally, with reference to FIG. 6D, dilator 52 may be pulled in a proximal direction to bring it to a fully retracted position within sheath 54. Delivery device 50 is then retrieved over guide wire 120 followed by retrieval of guide wire 120 from within the body. Transected end 124 of graft vessel 122 must then be closed which may be accomplished by tying it off with a suture or closing it with a clip.

End-to-Side Anastomosis

Referring now to FIGS. 7A-7C, an end-to-side anastomosis is described. Prior to loading the end-to-side anastomotic connector 110 within delivery assembly 50, the graft vessel 122 is operatively attached to it. Tubular member 114 of the device 110 is inserted into transected end 124 of graft vessel 122 and secured thereto by cooperating securement rings 116 and 118. Other securement means such as suture loops or the like may be used instead. The anastomotic connector 110 with attached graft vessel 122 is then loaded on dilator 52 by inserting tapered distal end 58 of dilator 52 into the proximal end (not shown) of graft vessel 122 until device 110 is fully contained within chamber 66 and graft 122 extends proximally over primary shaft portion 70 of dilator 52. Dilator 52 is then inserted into shaft 54. Alternately, graft vessel 122 or anastomotic connector 110 may be individually and independently loaded onto dilator 52 in either order and then interconnected as described above.

Next, guide wire 120 is inserted into the proximal end of guide wire lumen of dilator 52 and delivered to within the incision or arteriotomy site within the target vessel. Delivery device 50 is then delivered over the guide wire 120 to target vessel 130, at which point delivery device 50 is advanced such that the tapered distal end 58 of dilator 52 is caused to penetrate through the wall of the target vessel 130, as illustrated in FIG. 7A. Upon distal end 58 being fully inserted within target vessel 130, dilator 52 is advanced distally a selected distance through shaft 54 by pushing on end portion 84 of dilator 52 until end cap 86 is caused to abut stop surface 92 of dilator stop 88. Alternatively, sheath 54 may be retracted a selected distance in a proximal direction until stop surface 92 abuts end cap 86 of dilator of dilator 52. During such advancement or retraction, proper alignment of dilator 52 is ensured by aligning alignment strip 96 with alignment strip 94 of sheath cap 82. The distance advanced by dilator 52 or the distance retracted by sheath 54 causes a sufficient portion of chamber 66 to be exposed such that the flange member 112 of anastomotic connector 110 is allowed to deploy within target vessel 130, as illustrated in FIG. 7B.

Next, as shown in FIG. 7C, dilator 52 and sheath 54 are retracted in a proximal direction over guide wire 120, leaving graft vessel 122 attached to anastomotic connector 110 and anastomosed to target vessel 130. The transected end 124 of graft vessel 122 and the opening created in target vessel 130 are caused to be pulled together to maintain contact between their respective edges. Preferably, the endothelial linings of the vessels are in intimal contact with each other so as to promote natural tissue bonding between them. After complete removal of dilator/sheath assembly 50, guide wire 120 is retrieved from within the body. The proximal 124 of graft vessel 122 may then be anastomosed to a source of blood to complete the bypass.

Kits

Also provided are kits that include at least one anastomotic connector delivery device of the present invention, where in many embodiments the kits may include two or more delivery devices having varying dimensions so as to provide the physician convenience and security of having a device with the correct size for a particular patient. The kits may further include other tools such as proximator or sizing devices for determining the appropriate size of the device to be used, and the like, which devices find use in performing an anastomosis. The kit may further include one or more anastomotic connectors to be implanted having the same or different sizes, shapes and configurations. The subject kits may also include securing or reinforcement means, e.g., biocompatible glues/adhesives, hemostatic rings, clips, etc.

In addition, the subject kits typically include instructions for using the devices in methods according to the subject invention. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the Internet, are provided. An example of this embodiment is a kit that includes a web address where the instructions can be viewed and/or from which the instructions can be downloaded. As with the instructions, this means for obtaining the instructions is recorded on a suitable substrate.

It is evident from the above description and results that the subject invention provides important new devices and procedures for delivering and implanting anastomotic connectors which overcome a number of disadvantages currently encountered in the field of anastomosis. The subject delivery devices are easy to use and can provide for vessel joinder with out the use of sutures, staples, glues or other holding means. Moreover, the subject delivery devices are versatile and can be used in a variety of approaches and applications with a variety of differently configured connectors. As such, the subject invention represents a significant contribution to the field.

The instant invention is shown and described herein in what is considered to be the most practical, and preferred embodiments. It is recognized, however, that departures may be made there from, which are within the scope of the invention, and that obvious modifications will occur to one skilled in the art upon reading this disclosure.

What is claimed is:

1. An assembly for delivering an anastomotic connector having an expanded state and a constrained state, comprising:
a sheath having a lumen;
a dilator comprising a shaft selectively translatable within said lumen, said shaft having a recessed portion for operatively retaining said anastomotic connector thereon, wherein when said recessed portion is positioned within said shaft lumen, said anastomotic connector is in a constrained state.

2. The assembly of claim 1 wherein said recessed portion has a proximal end and a distal end, and is defined by a ledge at said proximal end.

3. The assembly of claim 2 wherein said recessed portion is defined by a proximally tapered shoulder at said distal end.

4. An assembly for delivering an anastomotic connector having an expanded state and a constrained state, comprising:
a sheath having a lumen;
a dilator comprising a shaft selectively translatable within said lumen and having a length and an annular space along a portion of said length wherein said annular space defines a cylindrical chamber when said annular space is positioned within said lumen for operatively retaining said anastomotic connector therein.

5. The assembly of 4 wherein said cylindrical chamber has a length in the range from about 8 to about 15 mm.

6. An assembly for delivering an anastomotic connector having an expanded state and a constrained state, comprising:
a sheath having a lumen;
a dilator translatable within said lumen and comprising a guide wire lumen extending the length of said dilator, a distal end portion having a tapered tip, a proximal shaft portion and a central shaft portion extending between said distal end portion and said proximal shaft portion, said central shaft portion having a diameter less than a diameter of said distal end portion and less than a diameter of said proximal shaft portion; and
a chamber defined by said central shaft portion when said dilator is in a fully retracted position within said sheath, said chamber dimensioned to operatively constrain said anastomotic connector.

7. The assembly of claim 6 further comprising means for aligning said dilator within said sheath to ensure proper deployment of said anastomotic connector.

8. The assembly of claim 7 wherein said alignment means comprises an alignment strip on said sheath and a corresponding alignment strip on said dilator.

9. A method for interconnecting a first vessel to a second vessel, comprising the steps of:
providing an anastomotic connector having a deployed state and a constrained state;
providing the assembly of claim 6;
operatively constraining said anastomotic connector within said chamber;
operatively engaging said first vessel with said assembly;
positioning said distal end portion of said assembly within said second vessel; and
deploying a first portion of said anastomotic connector within said second vessel.
deploying a second portion of said anastomotic connector within said first vessel; and
removing said assembly from said first and second vessels.

10. The method of claim 9 wherein said step of operatively engaging said first vessel with said assembly comprises inserting said distal end portion into a transected end of said first vessel and through a side wall of said first vessel.

11. The method of claim 10 wherein said step of deploying a first portion of said anastomotic connector comprises the step of advancing said dilator a selected distance in a distal direction.

12. The method of claim 11 wherein said step of deploying a second portion of said anastomotic connector comprises the step of advancing said dilator a second selected distance in a distal direction.

13. The method of claim 10 wherein said step of deploying a first portion of said anastomotic connector comprises the step of retracting said sheath a selected distance in a proximal direction.

14. The method of claim 13 wherein said step of deploying a second portion of said anastomotic connector comprises the step of retracting said sheath a second selected distance in a proximal direction.

15. The method of claim 9 wherein said step of operatively engaging said first vessel with said assembly comprises inserting said distal end portion of said dilator into a transected end of said first vessel and operatively positioning said first vessel about said proximal shaft portion of said dilator.

16. The method of claim 15 wherein said step of deploying a first portion of said anastomotic connector comprises the step of advancing said dilator a selected distance in a distal direction.

17. The method of claim 16 wherein said step of deploying a second portion of said anastomotic connector comprises the step of inserting said second portion into said first vessel prior to said step of operatively engaging said first vessel with said assembly.

18. The method of claim 9 wherein said step of positioning said distal end portion of said assembly within said second vessel comprises the steps of inserting a guide wire within said second vessel and delivering said assembly over said guide wire.

19. A method for forming a side-to-side connection between a first vessel and a second vessel, comprising the steps of:
providing a side-to-side anastomotic connector having a first member and a second member wherein each member has a constrained and deployed state;
providing the assembly of claim 6;
loading said anastomotic connector on said dilator;
constraining each said member within said chamber;
operatively engaging said first vessel with said assembly;
positioning said distal end portion of said assembly within said second vessel; and
deploying said first member of said anastomotic connector within said second vessel wherein the internal conduit pressure exerted on said first member causes said first member to form a substantially fluid-tight seal with an inner surface of said second vessel;
deploying said second member of said anastomotic connector within said first vessel; and
removing said assembly from said first and second vessels.

20. A method for forming an end-to-side connection between a first vessel and a second vessel, comprising the steps of:
providing an end-to-side anastomotic connector having a flange member and a tubular member;
providing the assembly of claim 6;
operatively connecting said tubular member with said first vessel;
loading said anastomotic connector on said dilator;
constraining said flange member within said chamber;
positioning said distal end portion of said assembly within said second vessel; and
deploying said first member of said anastomotic connector within said second vessel wherein the internal conduit pressure exerted on said first member causes said first member to form a substantially fluid-tight seal with an inner surface of said second vessel; and
removing said assembly from said first and second vessels.

21. A kit for interconnecting vessels, comprising:
at least one assembly of claim 6; and
at least one anastomotic device deliverable by said at least one assembly.

22. The kit of 21 further comprising instructions for using said assembly.
